(19) United States
(12) Patent Application Publication
(10) Pub. No.: US 2018/0296381 A1
(43) Pub. Date: Oct. 18, 2018

(54) GUIDE JOINT FOR A JOINT ORTHOSIS

(71) Applicant: Kurt WAYD, Wien (AT)

(72) Inventor: Kurt WAYD, Wien (AT)

(21) Appl. No.: 15/568,671

(22) PCT Filed: Apr. 20, 2016
(86) PCT No.: PCT/EP2016/058716

§ 371 Date: Oct. 23, 2017

(30) Foreign Application Priority Data
Apr. 22, 2015 (AT) A50323/2015

(51) Int. Cl. A61F 5/01

(52) U.S. Cl. CPC A61F 5/0125; A61F 2005/0148

(57) ABSTRACT

A guide joint (1) and a joint orthosis (2) provided with the guide joint for supporting and guiding an anatomical joint (7). The guide joint (1) has a first joint member (3) and a second joint member (4), and the joint members (3, 4) are connected to one another by a first linear guide (5) and a second linear guide (6). Each of the linear guides (5,6) has a guide track (10, 11) formed in the second joint member (4) as a slot or groove, in which in each case a sliding element (12, 13) arranged in the first joint member (3) engages, wherein the guide tracks (10, 11) intersect. The two sliding elements (12, 13) are spatially separate and spaced apart and protrude from a lateral surface of the first joint member (3).

The invention relates to a guide joint for a joint orthosis for physiological guidance of an anatomical joint, wherein the guide joint has a first joint member and a second joint member and the joint members are connected to one another by means of a first linear guide and a second linear guide, each of the linear guides having a guide track formed as a slot or groove in the second joint member, in which in each case a sliding element arranged on the first joint member engages, wherein the guide tracks intersect and the two sliding elements are spatially separate and spaced from one another and project from the first joint member.

The invention further relates to a joint orthosis for a knee joint with an upper leg fastening and a lower leg fastening, which are connected to one another by means of an outer guide joint and an inner guide joint.

The flexed motion of the human knee joint is a complex kinematic interaction of various translational and rotational motions, which are played out by the rolling off and sliding of the femoral condyles (medial and lateral condyles) of the upper leg bone (femur) on the opposing tibial plateau of the lower leg bone. The cohesion of the tibia and femur is ensured by ligaments, tendons, and muscles, which permit not only the flexed movement, but also a relative axial rotation as well as a lateral angular displacement of the tibia with respect to the femur (to a position comparable with bowleg or knock-knee).

In a complete flexion (thus a swiveling movement for an angle of 0°—an extended leg—to an angle of around 135° or more—a fully flexed leg), the upper leg does not only move about fixed axis with respect to the lower leg, but the upper leg also moves backward (posteriorly) with respect to the tibial plateau. This offset backward motion in an average-sized grown knee joint amounts to around 12-14 mm. In a full extension, thus a swiveling motion from an angle of 135° or more to an initial position of 0°, the lower leg (tibia) rotates, viewed from above, by around 15° about its own axis to the outside (laterally). At the same time, the tibia can also move laterally toward the femur in a small angular range, roughly comparable with a bowleg or knock-knee.

During the flexion the upper leg executes in the joint plane at the start, i.e. in the first 30° or so of the flexion, a nearly pure rotation about a fixed rotational axis and only then additionally executes a translational posterior and downward direction.

After knee operations, for a rapid healing process it is necessary above all to guide the knee joint in a targeted physiological manner. Even the flexing motion should be entirely possible, or if necessary with some angular restriction. This function is implemented by knee joint orthoses, which are structured from an upper leg fastening, a lower leg fastening, and a joint mechanism arranged between an upper and lower leg fastening, wherein the upper and lower leg fastenings are conventionally configured with bands or with hard shells.

Primarily the physiological guidance is only less satisfactorily achieved by many knee joint orthoses of the prior art.

In conventional knee joint orthoses, the joint mechanism consists for example of two monocentric rotational joints which are arranged on the opposing sides of the knee joint, and define a rotational axis through the knee joint. This rotational motion about a fixed rotational axis, however, contradicts the physiological kinematics of the anatomical knee. Since a rotation cannot take place simultaneously about two different axes, a difference motion occurs between the anatomical knee and the orthosis movement, which leads to withdrawals and displacements of the orthosis on the patient's leg, and is perceived by the patient primarily during more intense flexion of the knee as extremely uncomfortable. This difference motion can also have negative effects on the healing process, as the ligaments and tendons are additionally stressed.

Usually in the application of commercial knee orthoses, a “compromise range” is sought, which is selected such that during a complete flexion, the smallest displacements arise between the monocentric orthosis joint and the anatomical knee joint. The “compromise region” is a limited, small surface on the inside and outside of the knee, which best aligns with the variable range of rotation of the anatomical knee. This “compromise region” usually may be found in the posterior third of the femoral condyles. If the rotational axis is positioned outside of this limited surface, quite significant displacements and withdrawals of the orthosis on the patient's leg can occur. In practice, a consistently aligned mounting of the orthosis on the patient's leg can scarcely be achieved, however.

Numerous attempts to create physiologically correct joints for knee joint orthoses have failed due to the fact that the above-described complex motion processes of the knee joint, although adequately known, to date have not been adequately duplicated mechanically.

DE3504633 A1 discloses an orthotic knee joint arrangement in which two guide pins of a first joint part engage in recesses of a second joint part. The recesses limit the deflection movements of the joint, wherein the relative positions between the joint members with respect to the flexion angle are not unequivocally defined, but permit play.

DE473487 C discloses a knee joint for artificial legs with a double curved sliding guide, in which two guide pins of a joint part are guided in two associated guide grooves of the other joint part, said guide grooves intersecting with their rear ends, such that in the knee movement, the accordingly curved support edge on the lower end of the upper leg rail can slide along on a supporting roller of the lower leg rail.

WO2013040354 A1 discloses a knee joint orthosis, the joints of which have a fixed pivot point.

US 2013/0018293 A1, which was submitted by the present applicant, discloses several embodiments of joints for knee joint orthoses, with which it for the first time became possible to satisfactorily imitate the kinematic specifications of the knee joint. A joint embodiment disclosed in US 2013/0018293 A1 has two flat joint members contiguous to one another, wherein in one of the joint members a broad guide groove, running substantially longitudinally toward the member axis, and on the base of the guide groove, a narrow guide slot running substantially transversely to the member axis are provided. The other member axis has a circular plateau, the diameter of which corresponds to the width of the guide groove. Furthermore, on the plateau a bolt is arranged, the diameter of which corresponds to the width of the guide slot. The plateau is provided in the guide groove such that the bolt is arranged in the guide slot. By means of the two guides, arranged substantially transversely to one another, there arises between the two joint members a kinematic motion that very precisely corresponds to the knee movement. The translation motion of the upper joint member, thus the joint member that should execute the motion of the upper leg, here substantially corresponds to the length of the guide slot.

The complex manufacture of this joint is a disadvantage, however. Owing to the two contiguously arranged joint members, limiting pins, which are inserted in boreholes so as to angularly limit the flexing motion, are disadvantageously subject to bending.

It is the object of the present invention to provide a guide joint and a knee joint orthosis supplied with this guide joint, which further improves the prior art. In particular, a guide joint is to be created that is small and may be easily and simply manufactured. Between the first joint member and the second joint member, a correspond physiological joint should occur.

This and further goals are achieved according to the invention by means of a guide joint of the type named above, in which the first sliding element, during the first 30° of the joint motion, does not substantially change its position in the first guide track due to the forced guidance of the sliding elements in the guide tracks. This permits the manufacture of an economical and stable guide joint that can also durably resist static and dynamic loads as may occur in knee joint orthoses. The two rotational axes of the sliding elements are here parallel, but laterally offset with respect to one another. In this way, in the intersecting guide tracks, the position of the sliding elements is unequivocally defined in each flexion angle.

The spacing of the two rotational axes and the arrangement and length of the two guide tracks are selected such that the resultant motion during flexion of the guide joint as exactly as possible corresponds to the physiological flexion motion of a femoral head of average size.

In a preferred embodiment, the sliding elements can be arranged on two opposing sides of the first joint member. Here the sliding elements can protrude outwardly in different directions from the joint member or they can protrude inward from two opposing inner surfaces of the joint member in opposing directions.

In an advantageous embodiment, the first or second joint member can have boreholes for insertion of a limiting pin. In this way, the possible range of the flexion angle of the guide joint can be limited in a simple and effective manner. In a joint member that is configured from two side parts, the side parts can have congruent boreholes for insertion of a limiting pin.

In a further advantageous embodiment of the invention, the sliding surfaces can be provided on the sliding elements and/or on the guide tracks with a friction- and wear-reducing surface coating. A surface coating on the one hand reduces the wear of the guide joint, and can possibly replace laborious hardening.

The guide joint according to the invention can also be advantageously in prostheses.

The above-named joint orthosis for a knee joint, which is provided on both sides with a guide joint according to the invention, allows physiologically correct guidance of the joint over the entire range of flexion. Thanks to the attainable small size of the guide joints, the joint orthosis can have a very low weight and be well fitted to the shape of the body, so that in addition good wearing comfort is ensured.

Advantageously, here an outer upper leg rail of the upper leg fastening can be connected by a first hinge to the outer guide joint, and an inner upper leg rail of the lower leg fastening can be connected by a second hinge, an intermediate rail, and a third hinge to the inner guide joint. This allows advantageous fitting of the joint orthosis to different leg shapes. If necessary, depending on need, fewer or more than the above-listed hinges can be provided. The hinges can also allow a simple, space-saving folding of the unused joint orthoses.

In a preferred embodiment, the inner guide joint can be formed in a single piece with the inner upper leg rail and/or the outer guide joint can be formed in a single piece with the outer lower leg rail.

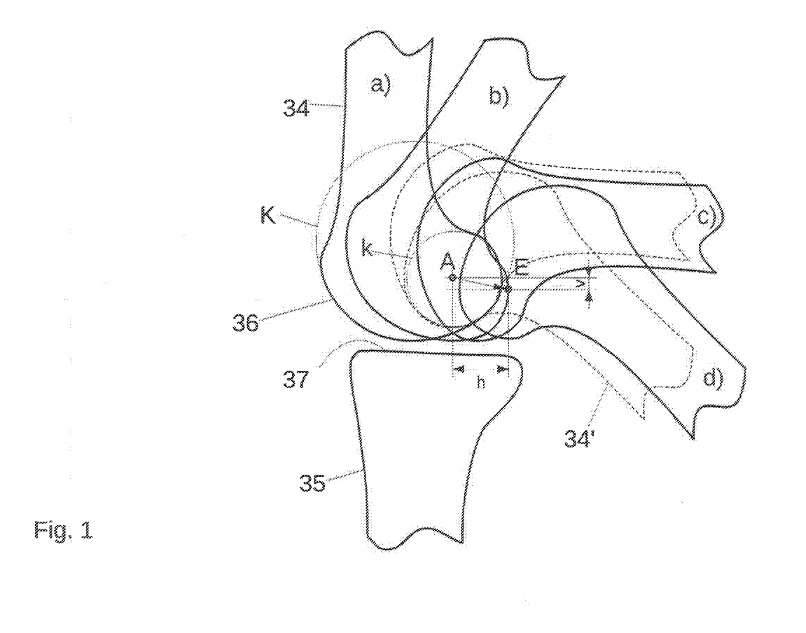

The present invention is elucidated in more detail below with reference to FIGS. 1 to 5, which show exemplary, schematic, and non-limiting advantageous embodiments of the invention. Wherein

FIG. 1 shows a schematic representation of the motion process of a human knee joint in a flexion motion of the knee;

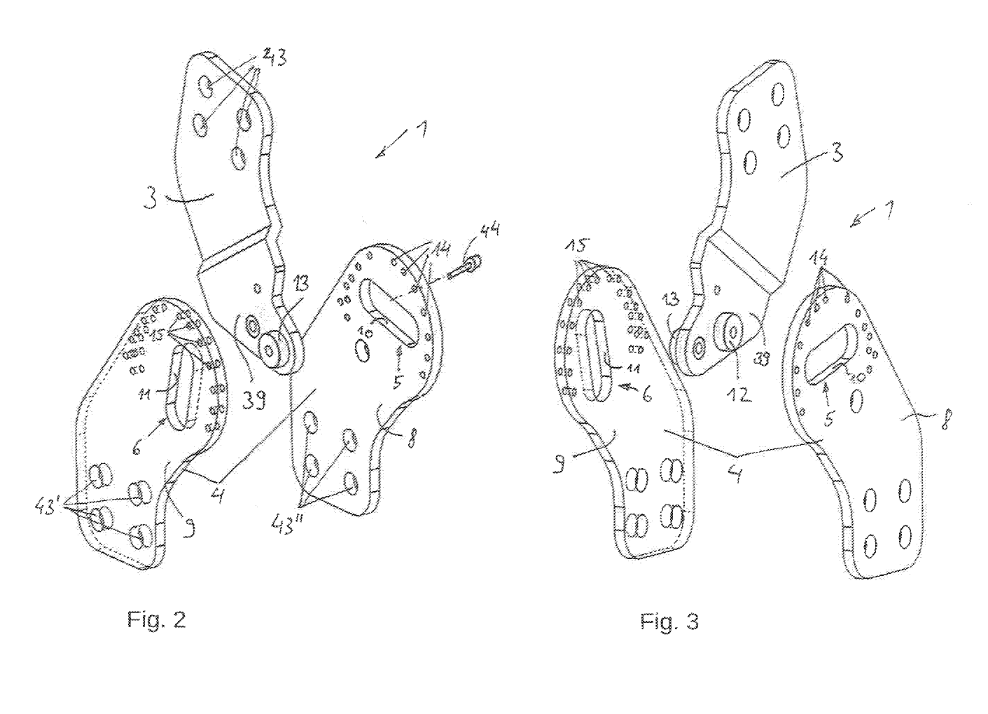

FIG. 2 shows a guide joint according to the invention in an exploded diagrammatic representation;

FIG. 3 shows the guide joint of FIG. 2 in a further exploded diagrammatic representation from a different perspective;

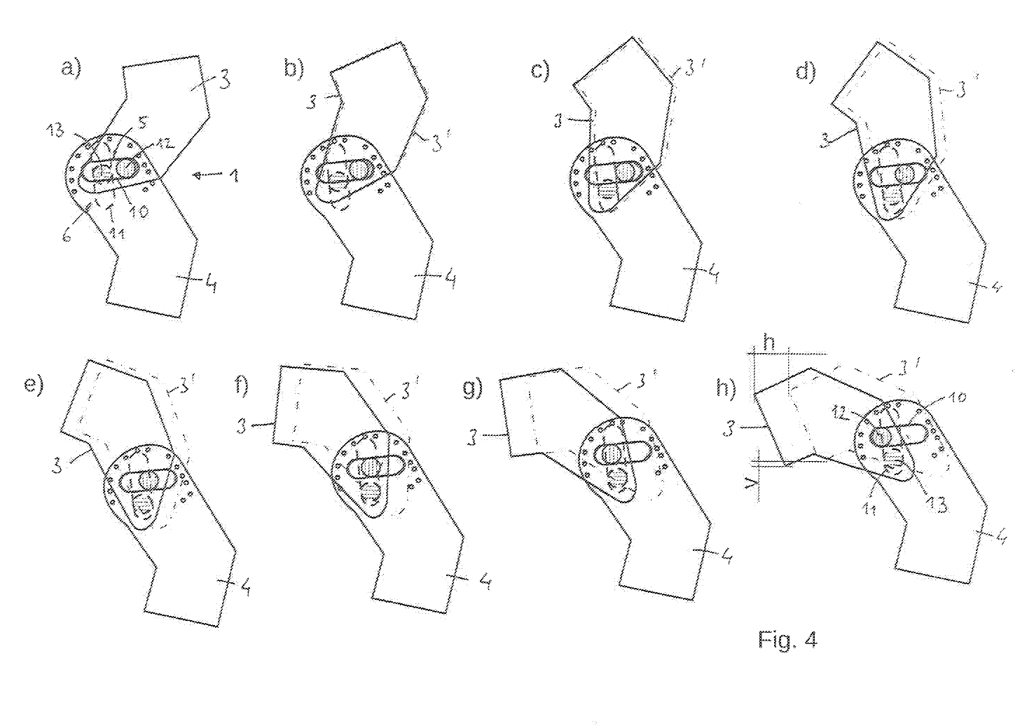

FIGS. 4a to 4h show a schematic representation of a motion process of the guide joint shown in FIGS. 2 and 3;

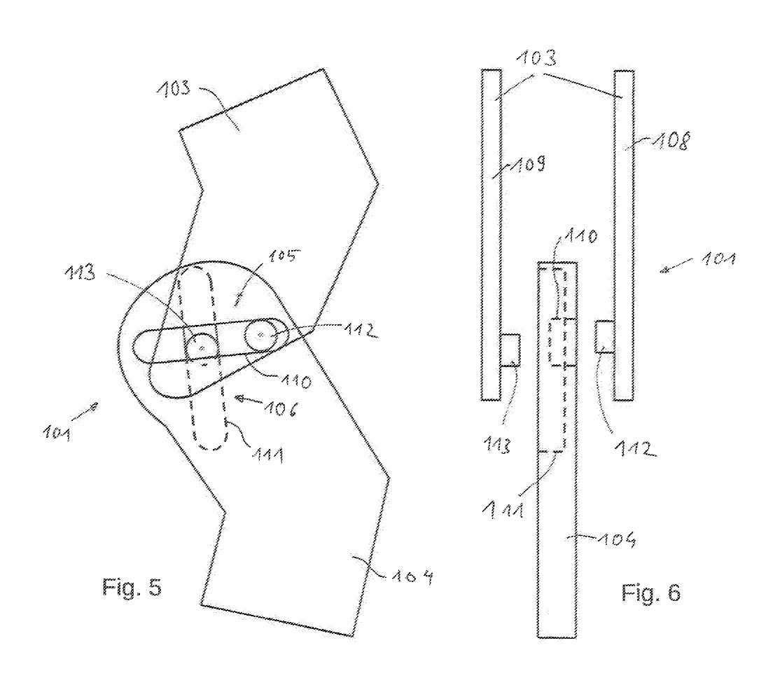

FIGS. 5 and 6 show an alternative embodiment of the guide joint according to the invention in two projections;

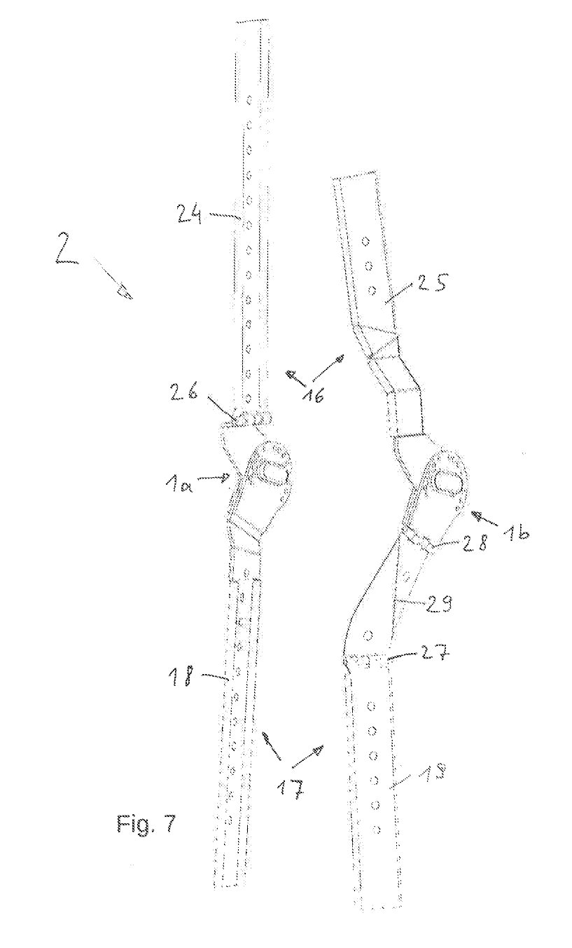

FIG. 7 shows two guide joints according to the invention with rails of a knee joint orthosis connected thereto; and

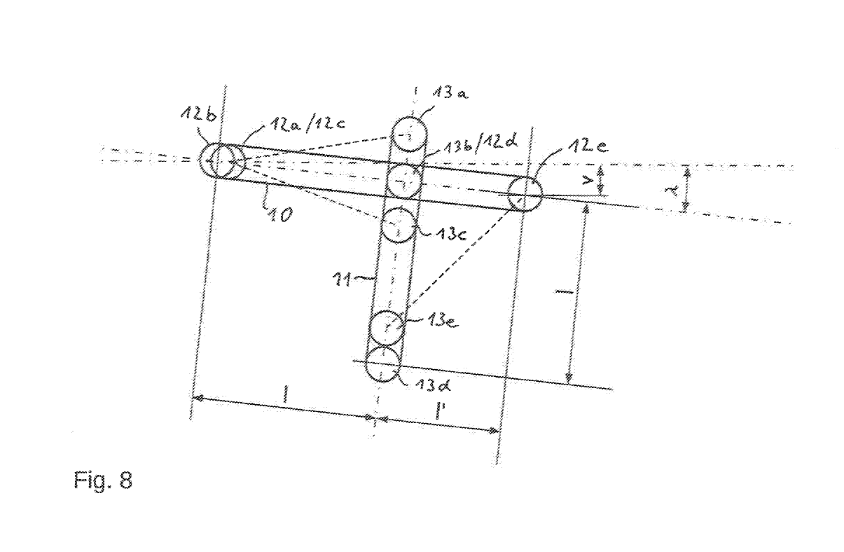

FIG. 8 shows a representation of the kinematic relationships with reference to motion of the sliding elements in their guide tracks during a flexion motion.

To illustrate the motion process that occurs in the flexion of a human knee joint, FIG. 1 shows this motion process with reference to four different angles of flexion of the knee, each in one sectional view through the femur 34 (bone of upper leg) and tibia (35) (shin), wherein the sectional plane corresponds to the joint plane which runs normally to the joint axis. For the sake of clarity, the other elements of the knee joint, such as for example the kneecap, the meniscus, the fibula, or the numerous ligaments are not shown in FIG. 1.

Proceeding from an extended leg (position a shows the tibial and femoral head at a flexion angle of 0°), the following positions b, c, and d each show a further flexed position of the knee joint at 30°, 90°, and 135°. A healthy knee joint can be flexed even further, wherein the maximal possible flexion generally is at an angle between around 140° to 150°.

During the flexion motion of the knee, the condyles 36 of the femur 34 slide on the abutting socket-like tibial plateau 37 of the tibia 35. Thus, the shape of the condyles 36 is important for the motion; their course in the joint plane can be exemplified in a simplified manner as two osculating circles K and k merging into one another, wherein the larger osculating circle K has a larger radius than the radius of the smaller osculating circle k. In the flexion motion, the condyles 36 slide initially, thus from the extended leg (0°) to a flexion of around 30°, nearly exclusively along the larger osculating circle K, so that theoretically for the femur 34 a nearly pure rotational motion about the midpoint of the larger osculating circle K is produced.

In fact the chosen pivot point for all monocentric joints is in a “compromise region,” which may be found in the posterior third of the femoral condyles, inside of the small osculating circle k.

From position b, a transition occurs from the large osculating circle K to the small osculating circle k, wherein the condyles on the tibial plateau in addition to the sliding motion also start to roll, and the center of rotation progressively displaces in a posterior direction. (This displacement corresponds to the displacement of a sliding element 12, described further below, along a guide track 10 of a linear guide 5 in FIGS. 2 to 4.)

The initial positions A of the variable center of rotation with respect to the tibial plateau changes during the flexion motion and wanders as far as an end position E, which in FIG. 1 is shown for the flexion angle of 135°. As can be seen from FIG. 1, the points of contact between the femoral condyles also change, and during the flexion motion wander significantly backward (posteriorly), which from a flexion angle of 30° may be explained by an additional rolling motion.

For the positions c (90° flexion) and d (135° flexion), the broken lines show the position of the femur 34′, which (proceeding from position a) would occur during a pure rotation of the femur 34 about a fixed rotational point, wherein the initial position A of the variable center of rotation was chosen as a virtual center of rotation. Here it can be seen that the actual position of the femur 34 in the position d with respect to the virtual position 34′ has been “displaced” by a horizontal displacement h and a vertical displacement v.

This horizontal and vertical displacement is the reason why a knee joint orthosis that has a pure rotational joint tends in the flexed position, starting from a flexion angle of 30°, to “push away” the upper leg from the lower leg, so that between the femur 34 and tibia 35, a tensile stress arises, which would additionally stress the ligaments that properly should be protected. In knee joint orthoses with a fixed rotational axis, therefore, usually the maximal rotational angle is limited. Here walking is still possible, but movements that require a greater flexion angle are impeded by the orthosis. A sports activity, for example running, bicycling, gymnastics, or swimming, is hardly possible with a limited maximal flexion angle. Also targeted training for muscle building and for recovery of full joint mobility following surgery cannot be satisfactorily carried out with the orthosis. Exercises that can require a greater flexion angle from the patient can no longer be done alone. Instead, the exercise must be carried out with the orthosis set aside, with the help of a therapist, who manually supports the joint during the exercise. With the help of an ergonomically correct orthosis, the patient could perform many exercises without the cost-intensive professional support by a therapist, more frequently and regularly.

In order to produce a physiological guide joint, therefore, the motion process shown in FIG. 1 must be copied as faithfully as possible, wherein in particular the attainment of the comparatively large horizontal displacement h of joints is not technically possible with a monocentric joint.

FIGS. 2 and 3 show a guide joint 1 according to the invention from two different perspectives. The guide joint 1 consists of a one-piece upper joint member 3 and a two-part lower joint member 4. The upper joint member 3 consists of a substantially flat plate, which has a guide region 39, from which, protruding from one side a first sliding element 12 and projecting from the opposing side a second sliding element 13 are arranged. The sliding elements 12, 13 are each formed as circular disks that are secured to the surface of the guide region 39. The disks can be fixed in place or can be formed as rollers, wherein the axes of the two sliding elements 12, 13 are arranged offset to one another. The two sliding elements are spatially separate from one another and, with regard to the joint plane, have no intersections. The surface of the sliding element 12, 13 has a high hardness and low friction, wherein this can be achieved either by hardening of the surface or by a coating.

The lower joint member 4 consists of a first side part 8 and a second side part 9, which have substantially the same outer contours. In the first side part 8, a slot-like first guide track 10 is provided for the first sliding element 12, and in the second side part 9 a slot-like second guide track 11 is provided for the second sliding element 13. The two sliding elements 12, 13, which each slide in a guide track 10, 11, form a linear guide 5, 6 with the latter on each side of the upper joint member 3. The guide tracks 10, 11 are formed as simple slots in the side parts 8,9, wherein simple or profiled grooves or other more complex guide tracks for the sliding elements 12, 13 of the upper joint member can also be provided. The sliding surfaces provided in the guide tracks 10, 11, as the corresponding surfaces of the sliding elements 12, 13, can be coated or hardened in order to improve the sliding properties and the durability of the joint.

The upper joint member 3 and the two lower side parts 8, 9 all have installation boreholes 43, with which the guide joint can be secured to the adjacent structures, for example to the upper leg fastening or the lower leg fastening of a knee joint orthosis. Instead of the installation boreholes 43, any desired types of fastening can be provided. Here a spacer element can be provided between the two side parts 8, 9, so that the space between the side parts 8, 9 corresponds to the thickness of the guide region 39 of the upper joint member 3 plus an additional play. Preferably for the lower joint member 4 the adjacent structure, perhaps a rail of the lower leg fastening, to which the guide joint is secured, can simultaneously perform the function of the spacer element.

Advantageously, the upper joint member 3 of the inner guide joint can be made of a piece of the upper leg fastening of a knee joint orthosis, so that the installation boreholes become unnecessary. This also applies to the outer guide joint, wherein a side part of the lower joint member 4 can be manufactured jointly with the lower leg fastening from a single piece. In this case of course the second side part must be provided with installation boreholes in order to hold the guide joint together.

Further, a number of boreholes 14, 15 are made in the side parts 8, 9 in the edge region, wherein a coaxial borehole 15 in the second side part 9 is assigned to each borehole 14 of the first side part 8, so that there are several borehole pairs. A limiting pin 44 can be inserted through borehole pair 14, 15 (shown in FIG. 2), which limits the motion of the upper joint member 3 to a specific angle. The limiting pin 44 can be provided with threading, for example, which is screwed into a threading provided in the borehole 15 in order to fix the limiting pin 44.

The designations “upper” and “lower” relate only to the alignment shown in FIGS. 2 and 3, but it is clear to a person skilled in the art that the joint can also be used inversely, so that the “lower” joint member 4 of FIGS. 2 and 3 can equally be built into a joint orthosis as an upper joint member. In this case the course of the linear guides 5, 6 must also be fitted accordingly. The terms “upper” and “lower” serve only to simplify the description and are not to be interpreted in a limiting manner. The same applies to the terms “inner” (thus, the side facing the knee joint) and “outer” (thus, the side facing away from the knee joint).

The two guide tracks 10, 11 are arranged in an intersecting manner with regard to the joint plane, wherein by means of the crosswise arrangement and the two sliding elements 12, 13 arranged offset from one another, an unequivocally specified relative position results for each flexion angle between the upper joint member 3 and the lower joint member 4. The first linear guide 5 is arranged substantially horizontally, wherein the posterior end, i.e. the end of the linear guide facing the inner side of flexion (thus for example the popliteal space) is arranged a little more deeply than the anterior end (thus, for example, the kneecap). This may be attributed to the fact that the midpoint of the small osculating circle k of the femoral condyles in the initial position at 0° flexion is further from the tibial plateau than in the end position at 135°. The second linear guide 6 is normally aligned on the first linear guide 5, that is, substantially vertically. The angle between the first and second linear guide 5, 6 is around 90°, but the linear guides can also be arranged at acute or obtuse angles to one another.

The relative motion during flexing of the guide joint 1 is shown in FIGS. 4a to 4h, wherein the essential elements of the guide joint 1 are projected onto the joint plane. Here in particular the forcibly guided motion of the sliding elements 12, 13 fixedly connected to the upper joint member 3 in the crosswise arranged guide tracks 10, 11, should be noted. In the positions of FIGS. 4a to 4c, which correspond to a flexion of around 20°, 35°, and 40°, respectively, the first sliding element 12 remains in the region of the anterior end of the first guide track 10, while the second sliding element 13 moves backwards in the second guide track 11. The motion of the upper joint member 3 (with respect to the fixed lower joint member 4) here corresponds substantially to a swinging motion about the first sliding element 12, which in this region of the flexion motion can be viewed substantially as a fixed joint axis. The flexion motion corresponds substantially to the physiological motion section in an anatomical knee joint, in which the condyles 36 slide along the first osculating circle K (cf. FIG. 1).

Starting with a flexion angle of 30° of the position shown in FIG. 4c, and intensified in FIGS. 4d to 4e, with further flexion of the guide joint 1, the first sliding element 12 starts to move backwards (posteriorly, i.e., away from the anterior end of the guide track 10). At a flexion angle of 90°, the second sliding element 13 has reached the deepest point in the guide track 11 (lower end of the guide track 11) and with further flexion again moves upward until the first sliding element 12 with full flexion is at the posterior end of the first guide track 10. With subsequent extension, the second sliding element 13 again moves downwards, wherein at a flexion angle of 90° it again reaches the deepest point and with further extension it again moves all the way upward to the initial position at 0° (the sliding element 13 oscillates at full flexion and extension between 90° and 135°).

In order to hold the pivot point in a fixed position during the first 30°, the second sliding element 13 was placed in an angle of 15° above the sliding element 12 in the second guide track 11. If now the sliding element 13, which is, along with the sliding element 12, on the joint member 3 assigned to the upper leg, rotates in an arc of 15° about the sliding element 12, both sliding elements are then on the axis of the guide track 10. But since the guide track 11 over the first 15° does not describe a circle but a straight line, and the separation of the midpoints of the two sliding elements is greater than the separation from the first sliding element 12 to the interface of the two guide tracks, the first sliding element 12 is pushed slightly outward. The guide track 10 must be extended by this difference measure so as to avoid blocking of motion. With rotation of another 15°, the first sliding element 12 again migrates back by this difference measure to the initial position. Now the joint member 3 has executed a rotation of 30° without the first sliding element 12, which constitutes the mobile center of rotation, substantially altering its position.

The linear guide 6 (guide track 11) could also run in a curve, however, so that at the start of the flexion motion (with a flexion between 0° and around 30°), a pure circulation about the first sliding element 12 could result. The linear guide 6 toward this end would have to be guided in an arc of 30° about the anterior end of the first linear guide 5, and subsequently again run straight. However, the embodiment shown with straight-running sliding elements 12, 13 suffices to copy a physiologically correct flexion motion adequately well (the differential motion of the sliding element 12 during the first 30° is insignificant and can be ignored).

To illustrate the flexion motion that the upper joint member 3 makes with respect to the fixed lower joint member 4, in comparison with a pure swinging motion about a fixed axis, FIGS. 4b to 4h indicate with dot-dash lines a position of a comparison upper joint member 3′, which would result if this comparison upper joint member 3′ proceeding from the position shown in FIG. 4a were to rotate about a fixed pivot axis. As the fixed pivot axis of the comparison upper joint member 3′, the position of the first sliding element 12 shown in FIG. 1a was chosen. In particular in FIG. 4h it can be seen that the guide joint 1 according to the invention during the flexion motion has both a horizontal displacement h and a vertical displacement v, which substantially correspond to the horizontal displacement h and the vertical displacement v of the anatomical knee joint, as shown in FIG. 1.

FIG. 8 shows the forced guidance of the sliding elements 12, 13 in the guide tracks 10, 11 during a complete flexion motion (0° to 135°), wherein the reference symbols of the sliding elements 12, 13 in the individual shown positions are supplemented according to their sequence by the lowercase letters a, b, c, d, and e.

It can be seen from FIG. 8 how the arrangement of the guide tracks according to the invention effects a forced guidance, in which the position of the first sliding element during the first 30° remains substantially unchanged. In the motion process thereby achieved, in the first 30° of flexion the joint executes a substantially pure rotational motion about the first sliding element 12, after which the sliding element moves progressively backward.

Due to the guide tracks crossed under 90°, it can be ensured that the first sliding element 12 returns after around 30° flexion to the same position 12c, which also defines the starting point 12a of the flexion (0°). Here the sliding element executes only a slight deflection movement (to position 12b), which is negligible in relation to the entire motion process.

The second sliding element 13 during the first 30° of flexion moves from the initial position 13a through position 13b to position 13c, which corresponds to more than a third of the entire track length of the first guide track 11. The first sliding element 12 on the other hand changes its position only insignificantly, wherein it executes only a minimal deflection from the initial position 12a (0°) to 12b (15°) and again back to 12c (30°), which is practically in the millimeter range and corresponds to only a fraction of the length of the second guide track 10. This deflection occurs due to the straight course of the first guide track and can be practically ignored due to its short extent. Thus, for the first sliding element 12 during the first 30° of the flexion motion there is a change of direction in the guide track, while during this first 30° of the flexion, the second sliding element 13 moves in only one direction in the guide track.

Only with further motion of the second sliding element 13 downwards (to positions 13d and 13e) does the first sliding element 12 execute a progressively increased motion backwards to the positions 12d (at the point of intersection of the two guide tracks 10, 11) and 12e (at the posterior end of the guide track 10). This loitering of the first sliding element 12a, 12b, and 12c in the initial position during the first 30° of the flexion motion (or the return of 12c of the first sliding element to the initial position 12a after the first 30° of the flexion motion) and the subsequently progressively increased motion of this sliding element backwards (12d, 12e) until maximal flexion (around 135°) ensures a flexion motion that is optimally perceived as the flexion motion of an anatomical knee joint.

As is shown in FIG. 8, the first guide track 10 is obliquely arranged by an angle λ with respect to the horizontal, so that the posterior end of the guide track is arranged more deeply by a vertical displacement v with respect to the anterior end. The angle λ is preferably only around 3°-5°, but this is adequate in order to readily copy the initially described displacement of the femur of the anatomical knee joint.

The length ratio of the anterior length l of the first guide track 10 (thus, the length l that is in front of the point of intersection of the guide tracks) to the posterior length l′ of this guide track 10 in the case shown is around 1:0.707. This occurs when the arrangement of the guide tracks is below an angle of 90 degrees, under the secondary conditions that the maximal flexion angle is 135° and that the first sliding element returns to its initial position (12a or 12c) after a flexion motion of 30°. The length l here corresponds to the distance between the first and second sliding elements.

FIGS. 5 and 6 schematically illustrate a further embodiment of a guide joint 101 according to the invention, in which the linear guides 105, 106 were “reversed” in comparison with the embodiment shown in FIGS. 2 to 4, wherein the sliding elements 112, 113 on two opposing side parts 108, 109 of the upper joint member 103 are arranged to protrude inward. The two intersecting guide tracks 110, 111 of the linear guides 105, 106 are formed as grooves in the lower joint member 104 disposed between the side parts 108, 109. In order to be able to reduce the thickness of the lower joint member 4, the grooves of the guide tracks 110, 111 intersect in their crossing region.

The flexion motion of the guide joints 1, 101 shown in FIGS. 2 to 6 allow manufacture of anatomically correct knee joint orthoses 2, which permit the therapist to set the specifications for a maximal (and possibly minimal) flexion angle on the basis of the medical indication, rather than on the basis of the inadequacy of the knee joint orthosis 2 used. The use of such a guide joint in a knee joint orthosis 2 is illustrated in FIG. 7.

FIG. 7 shows the elements of the upper leg fastening 16 and the lower leg fastening 17 of the knee joint orthosis 2, which are connected guide joints 1a and 1b to be assigned to the two sides of the knee joint. The outer guide joint 1a connects an outer upper leg rail 24 to an outer lower leg rail 18, wherein advantageously the connection to the upper leg fastening is by means of a hinge. The inner guide joint 1b connects an inner upper leg rail 25 to an inner lower leg rail 19, wherein the connection to the lower leg rail is through two hinges, which are connected to one another through an intermediate rail. The upper leg fastening 16 and the lower leg fastening 17 can each be provided with fastening elements, for instance bands, for fastening to the upper or lower leg, or can be secured with known hard shells (not shown in FIG. 7).

### REFERENCE SYMBOLS

guide joint 1
knee joint orthosis 2
upper joint member 3
lower joint member 4
first linear guide 5
second linear guide 6
anatomical joint 7
side parts 8, 9
guide tracks 10, 11
sliding elements 12, 13
boreholes 14, 15
upper leg fastening 16
lower leg fastening 17
outer lower leg rail 18
inner lower leg rail 19
outer upper leg rail 24
inner upper leg rail 25
first, second, third hinge 26, 27, 28
intermediate rail 29
femur (bone of upper leg) 34
tibial (bone of lower leg) 35
condyle 36
tibial plateau 37
marking point 38
guide region 39
installation borehole 43, 43′, 43″
limiting pin 44

1. A guide joint (1) for a joint orthosis (2) for supporting and guiding an anatomical joint (7), wherein the guide joint (1) has a first joint member (3) and a second joint member (4), and the joint members (3, 4) are connected to one another by a first linear guide (5) and a second linear guide (6), wherein each of the linear guides (5,6) has a guide track (10, 11) formed in the second joint member (4) as a slot or groove, in which in each case a sliding element (12, 13) arranged in the first joint member (3) engages, wherein the guide tracks (10, 11) intersect, the two sliding elements (12, 13) are spatially separate and spaced apart from one another and protrude from the first joint member (3), wherein the first sliding element (12) during the first 30° of the joint flexion does not substantially change its position in the guide track (10) due to the forced guidance of the sliding elements (12, 13) in the guide tracks (10, 11).

2. The guide joint (1) according to claim 1, wherein the two guide tracks (10, 11) are arranged to intersect one another at an angle of 90° relative to the joint plane.

3. The guide joint (1) according to claim 1, wherein the first or second joint member (4) has two side parts (8, 9, 108, 109) that are connected to one another.

4. The guide joint (1) according to claim 3, wherein the sliding elements (12, 13, 112, 113) are arranged on two opposing sides of the first joint member (3, 103).

5. The guide joint (1) according to claim 3, wherein the first and second joint members (3, 4) have boreholes (14, 15) for insertion of a limiting pin (44).

6. The guide joint (1) according to claim 1, wherein that sliding surfaces on the sliding elements (12, 13) and/or on the guide tracks (10, 11) are provided with a friction- and wear-reducing surface coating.

7. A joint orthosis (2) for a knee joint with an upper leg fastening (16) and a lower leg fastening (17), wherein the upper leg fastening (16) has an outer guide joint (1a) and an inner guide joint (1b), each of which is connected to the lower leg fastening (17) in accordance with claim 1.

8. The joint orthosis (2) according to claim 7, wherein an outer upper leg rail (24) of the upper leg fastening (16) is connected to the outer guide joint (1a) by a first hinge (26) and that an inner lower leg rail (19) of the lower leg fastening (17) is connected to the inner guide joint (1b) by means of a second hinge (27), an intermediate rail (29), and a third hinge (28).

9. The joint orthosis (2) according to claim 7, wherein the inner guide joint (1b) is formed in a single piece with the inner upper leg rail (24) and/or the outer guide joint (1a) is formed in a single piece with the outer lower leg rail (18).
